(19) United States
(12) Patent Application Publication
(10) Pub. No.: US 2015/0073391 A1
(43) Pub. Date: Mar. 12, 2015

(54) MEDICAL DEVICE WITH A MOVABLE TIP

(71) Applicant: Boston Scientific Scimed, Inc., Maple Grove, MN (US)

(72) Inventors: John Hutchins, North Attleboro, MA (US); Robert B. DeVries, Northboro, MA (US); John A. Griego, Blackstone, MA (US)

(21) Appl. No.: 14/483,844

(22) Filed: Sep. 11, 2014

Related U.S. Application Data
(60) Provisional application No. 61/877,132, filed on Sep. 12, 2013.

(51) Int. Cl. A61M 25/09

(52) U.S. Cl. CPC A61M 25/09; A61M 25/09041; A61M 2025/09175; A61M 2025/09116; A61M 2210/1075; A61M 2210/10
USPC 604/528

(57) ABSTRACT

Medical devices and methods are disclosed. An example medical guidewire for accessing a body lumen along a biliary and/or pancreatic tract may include an elongated member having a distal end and a proximal end. The guidewire may include a movable distal tip positioned at the distal end of the elongated member. The guidewire may also include an electromechanical actuator for actuating movement of the distal tip. The actuation of the electromechanical actuator actuates movement of the adjustable distal tip and facilitates cannulation of one or more of a bile duct and a pancreatic duct.

### CROSS-REFERENCE TO RELATED APPLICATIONS

This application claims the benefit of U.S. Provisional Application No. 61/877,132, filed Sep. 12, 2013, the entire disclosure of which is incorporated herein by reference.

### TECHNICAL FIELD

The present disclosure pertains to medical devices, and methods for manufacturing and use of these medical devices. More particularly, the present disclosure pertains to medical devices for accessing a body lumen along a biliary and/or pancreatic tract.

### BACKGROUND

A wide variety of intracorporeal medical devices have been developed for medical use, for example, for endoscopic procedures. Some of these devices include guidewires, catheters, catheter systems, endoscopic instruments, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices and medical systems.

In one aspect, the present disclosure provides a medical guidewire for accessing a body lumen along a biliary and/or pancreatic tract. The guidewire may include an elongated member having a distal end and a proximal end. A movable distal tip may be positioned at the distal end of the elongated member. The guidewire may also include an electromechanical actuator for actuating movement of the distal tip. The actuation of the electromechanical actuator may actuate movement of the adjustable distal tip and facilitate cannulation of one or more of a bile duct and a pancreatic duct.

In another aspect, the present disclosure provides a medical device for use with an endoscope for accessing a body lumen along a biliary and/or pancreatic tract. The medical device may include an elongated member having a proximal end, a distal end, and a lumen defined therein. An enabled distal tip may be disposed at the distal end of the elongated member. An actuator element may be in mechanical communication with the distal tip to enable movement of the distal tip. The medical device may also include a control mechanism in electrical communication with the actuator element. The control mechanism may be capable of effecting mechanical movement of the actuator element. Adjustment of the control mechanism may adjust movement of the distal tip.

In another aspect, the present disclosure provides a method for accessing a body lumen along a biliary and/or pancreatic tract using a guidewire. The guidewire may have an electromechanical actuator capable of actuating movement of a distal tip of the guidewire. The guidewire may have an electromechanical actuator in communication with a distal tip of the guidewire. The guidewire may be advanced through a body lumen to a location where a common duct splits into a first duct and a second duct. The electromechanical actuator may be actuated to effect movement of the distal tip of the guidewire adjacent the first duct. The guidewire may be advanced into the first duct.

The above summary of some embodiments is not intended to describe each disclosed embodiment or every implementation of the present disclosure.

### BRIEF DESCRIPTION OF THE DRAWINGS

The disclosure may be more completely understood in consideration of the following detailed description in connection with the accompanying drawings, in which:

FIG. 1 is a schematic overview of the biliary tree;

FIG. 2 is a schematic side view of a portion of an illustrative guidewire according to an aspect of the present disclosure;

FIG. 3 is a schematic cross-sectional side view of a portion of an illustrative guidewire according to an aspect of the present disclosure;

FIG. 4 is a schematic cross-sectional side view showing a portion of an illustrative guidewire according to an aspect of the present disclosure;

FIG. 5 is a schematic view of illustrative movements of a distal tip of an illustrative guidewire according to an aspect of the present disclosure;

FIG. 6 is a schematic view of illustrative movements of the distal tip of an illustrative guidewire according to an aspect of the present disclosure;

FIG. 7 is a schematic view of illustrative movements of the distal tip of an illustrative guidewire according to an aspect of the present disclosure;

FIG. 8 is a schematic cross-sectional side view of a portion of an illustrative guidewire according to an aspect of the present disclosure;

FIG. 9 is a schematic cross-sectional side view of a portion of an illustrative guidewire according to an aspect of the present disclosure;

FIG. 10 is a schematic cross-sectional side view of a portion of an illustrative guidewire according to an aspect of the present disclosure;

FIG. 11 is a schematic cross-sectional side view of a portion of an illustrative guidewire with pull wires according to an aspect of the present disclosure;

FIG. 12 is a schematic cross-sectional side view of a portion of an illustrative guidewire with pull wires according to an aspect of the present disclosure; and

FIG. 13 is a schematic flow diagram of an illustrative method of using a guidewire according to an aspect of the present disclosure.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with one embodiment, it should be understood that such feature, structure, or characteristic may also be used in connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

As discussed herein, it may be desirable for a distal tip of a medical device (e.g., a guidewire) to be flexible to navigate effectively through a body lumen. For example, flexible distal tips of guidewires may be capable of facilitating navigation through narrow passages such as the papilla of Vater and/or other passages. In some instances, a flexible distal tip of a guidewire may facilitate steering the guidewire into a target body lumen that is closely situated to structures such as lesions, stones or other build-up and/or has such structures situated therein.

In some instances, the devices and methods that are disclosed herein may be useful for diagnostic or therapeutic procedures in the biliary and/or pancreatic tracts, among being useful for other purposes. Access to the pancreaticobiliary system, as facilitated by the devices disclosed herein, may be required to diagnose and/or treat a variety of conditions, including but not limited to tumors, gallstones, infection, sclerosis, and pseudo cysts. The device disclosed herein may also be useful for navigation in other parts of the body such as the cardiovascular system and so forth.

Endoscopic retrograde cholangio pancreatography (ERCP) may be used to diagnose and treat conditions of the common bile duct, including, for example, gallstones, inflammatory strictures, leaks (e.g., from trauma, surgery, etc.), and cancer. In an ERCP procedures, through an endoscope, a physician may view the inside of the stomach and/or the duodenum. Often, dyes may be injected into the ducts in the biliary tree and pancreas so that the area can be seen using X-rays. These procedures may necessitate gaining and keeping access to the papilla of Vater, the common bile duct, and/or the pancreatic duct, which may be technically challenging, may require extensive training and practice to gain proficiency, and may require one or more expensive tools in order to perform.

During an ERCP procedure, a number of steps are typically performed while the patient is often sedated and/or anaesthetized. For example, an endoscope may be inserted through the mouth, down the esophagus, into the stomach, through the pylorus into the duodenum, to a position at or near the papilla of Vater (also referred to as the ampulla of Vater), which is the opening of the common bile duct and the pancreatic duct. Due to the shape of the papilla, and the angle at which the common bile and pancreatic ducts meet the wall of the duodenum, the distal end of the endoscope is generally placed just past the papilla. Due to the positioning of the endoscopes beyond the papilla, the endoscopes typically used in these procedures are usually side-viewing endoscopes. The side-viewing feature provides imaging along the lateral aspect of the tip rather than from the end of the endoscope. Such orientation may allow a clinician to obtain an image of the medial wall of the duodenum, where the papilla of Vater is located, even though the distal tip of the endoscope is beyond the opening.

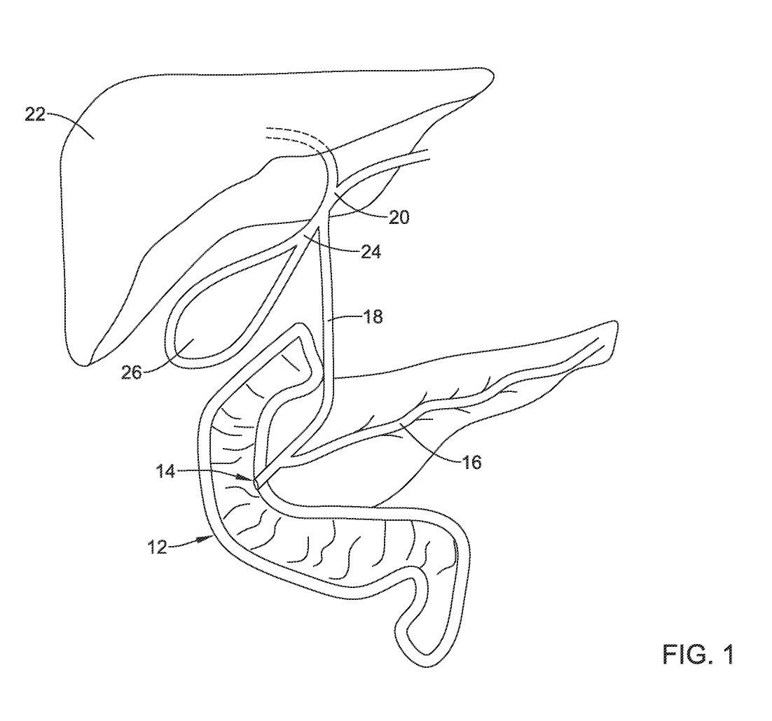

FIG. 1 illustrates an overview of the biliary system or tree. The papilla of Vater 14 is located in a portion of the duodenum 12. For the purpose of this disclosure, the papilla of Vater 14 is understood to be of the same anatomical structure as the ampulla of Vater. The papilla of Vater 14 generally forms the opening where the pancreatic duct 16 and the common bile duct 18 empty into the duodenum 12. The hepatic ducts, denoted by the reference numeral 20, are connected to the liver 22 and empty into the common bile duct 18 (also referred to as the bile duct). Similarly, the cystic duct 24 is connected to the gall bladder 26 and also empties into the common bile duct 18. In general, an endoscopic or biliary procedure may include advancing a medical device to a suitable location along the biliary tree and then performing the appropriate intervention.

Accessing a desired target along the biliary tree involves advancing the endoscope through the duodenum 12 to a position adjacent to the papilla of Vater 14, and advancing a medical device, which may be a guidewire, through the endoscope and through the papilla of Vater 14 to the intended target. The intended target may be, for example, the pancreatic duct 16 or the common bile duct 18.

The physician or clinician may advance the catheter through the papilla 14 and then attempt to advance the guidewire into the intended target duct. Sometimes, however, the clinician may end up inadvertently advancing the guidewire (and/or catheter) into an undesired duct. When the guidewire advances into the “undesired” duct, the clinician may be required to retract and advance the guidewire to a desired duct until the guidewire reaches the desired duct. This recurring procedure of retracting and advancing the guidewire may cause damage to surrounding tissue. Alternatively, the clinician may choose to pull the catheter from the body while leaving the guidewire in the non-target duct and then replace the catheter (or advance a new catheter) and load a second guidewire through the catheter to access the “desired” target duct. Such a technique may improve the chances of accessing the desired duct, for example, because the initial guidewire may partially block the “undesired” duct. Each of these procedures, however, may include removal of the catheter from the biliary tree and subsequent steps may involve re-cannulation of the papilla of Vater 14 (e.g., insertion of the medical device through the papilla). In addition, repeated cannulation of, for example, the common bile duct 18 and/or the pancreatic duct 16 may cause undesired side effects such as irritation or inflammation of tissue in the ducts and post-ERCP complications such as pancreatitis.

Further, several factors may complicate the cannulation of the papilla of Vater 14 such as an irregular sphincter orientation, floppy or irregular intraductal segments, variations of the biliary or pancreatic take-off levels, presence of stones or strictures in the lumen, and/or inflammation of the common bile or the pancreatic ducts. Difficult cannulations carry a high risk of perforation or other damage to tissue.

In one example procedure, physicians may use a technique for cannulation which involves identification of a bile trail by pushing against the papilla or applying suction to encourage bile to be released from the papilla. Prolonged probing and/or suction, however, may lead to adverse effects such as inflammation of the papilla. Thus, there is a need to develop medical devices that may facilitate cannulation of the papilla without causing harm to the tissue and/or the papilla.

Disclosed herein are example medical devices such as medical guidewires that may improve access to the desired location along the biliary tree. In general, these devices and methods may allow a catheter, guidewire, or the like to successfully access a target location along the biliary tree (e.g., the common bile duct 18 and/or the pancreatic duct 16).

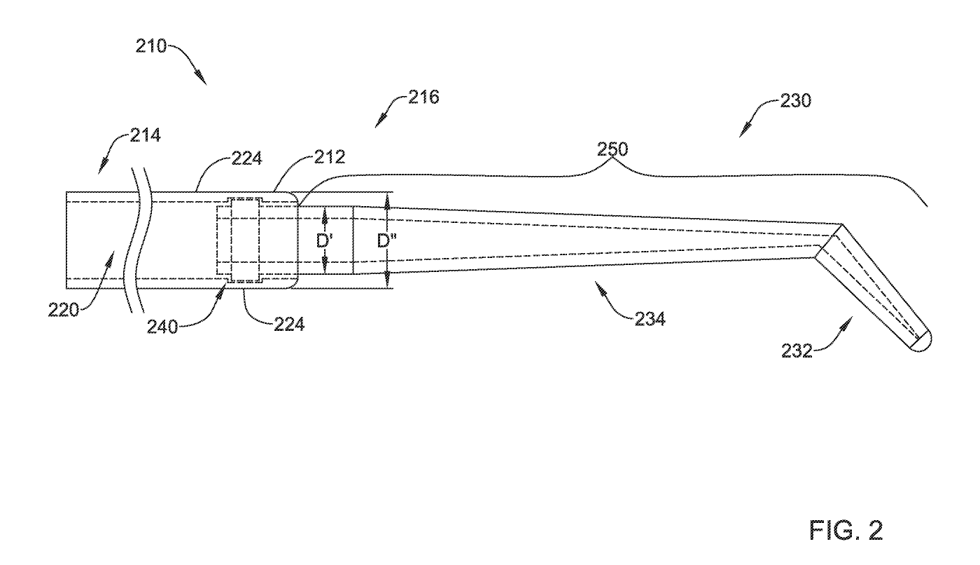

FIG. 2 illustrates a portion of an example medical guidewire 210. The guidewire 210 may include a shaft or elongated member 212 having a proximal end 214 and a distal end 216. The elongated member 212 may have a lumen 220 extending longitudinally from the proximal end 214 to the distal end 216. The distal end 216 of the guidewire 210 may also include a distal tip 230 that may be connected to the distal end 216.

The elongated member 212 may be unitarily formed (e.g., monolithic) or formed of two or more interconnected features, members, and/or components. As shown in FIGS. 2-4 and 8-11, the elongated member 212 may be formed of at least a main body 224 and a distal tip 230, where the lumen 220 extends therethrough. The elongated member 212 may have any dimensions as desired to facilitate travel through body lumens. In one example, the main body 224 may have a maximum diameter length D′ and the distal tip 230 may have a maximum diameter length D″, where the maximum diameter length D″ is less than the maximum diameter length D′.

In some instances, as shown in FIG. 2, the distal tip 230 of the elongated member 212 may be an assembly of smaller components. The distal tip 230 may include a body 234 and a deflectable tip 232. In some instances, the body 234 and/or the deflectable tip 232 may be tapered to facilitate traversal of the distal tip 230 through narrow openings. Alternatively, the body 234 and/or the deflectable tip 232 may have uniform diameters throughout their lengths. The shape of the distal tip 230 may be designed to correspond with the anatomy of the body lumen that is being accessed.

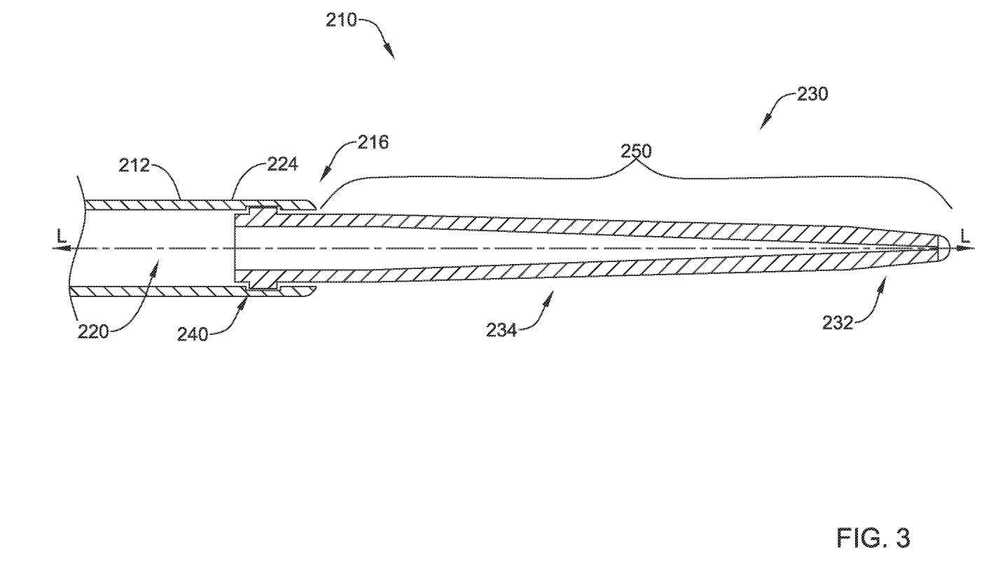

The deflectable tip 232 may be configured to bend and/or rotate at an angle from an undeflected position along longitudinal axis L-L (see FIG. 3). The deflectable tip 232 may be freely bendable and/or rotatable with respect to the body 234 of the distal tip 230.

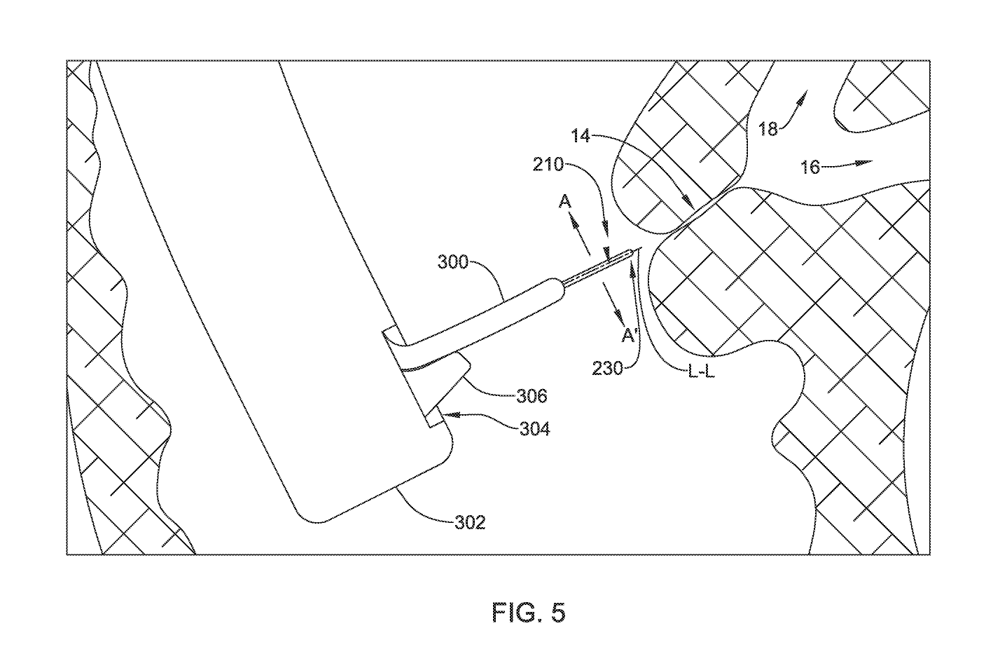

In some instances, the entire or substantially entire distal tip 230 may be configured to be moved and/or steered to access a target body lumen. Illustratively, the distal tip 230 may move independent of the main body 224 of the elongated member 212. Also, the distal tip 230 may be capable of and/or configured to undergo different motions, for example, vibration motions (e.g., side-to-side with respect to a longitudinal axis L-L), rotation motions (e.g., concentric or substantially concentric motion about the longitudinal axis L-L), longitudinal oscillation (e.g., in and out axial movement along the longitudinal axis L-L), etc. to facilitate access to and/or through the target body lumen. In some instances, the entire or substantially entire guidewire 210 may undergo different motions and/or may be steered or, alternatively, a portion (e.g., proximal end 214, the mid-portion 215, the distal end 216, etc.) of the guidewire 210 may undergo different motion and/or may be steered. Illustrative motions of the distal tip 230 and/or the guidewire 210 will be discussed infra with reference to FIGS. 5-7.

In some instances, a number of slots (not shown) may be provided on or at one or more portions of the guidewire 210 (e.g., a portion 250 of the guidewire 210) to impart flexibility to the distal tip 230, thereby enabling the distal tip 230 to be further movable and/or steerable. Illustratively, the slots may be arranged circumferentially and along a longitudinal axis of the distal tip 230. In some embodiments, the slots may be provided on an outer surface of the elongated member 212, thereby imparting flexibility in movement of the elongated member 212. Detailed description of the slots will be discussed infra.

In some instances, the distal tip 230 may be mechanically coupled to the main body 224 of the guidewire 210 at a connection 240, as shown in FIG. 2. Details of such a mechanical coupling will be discussed in conjunction with subsequent figures.

The distal tip 230 may be made from biocompatible materials such as polymers, Nitinol (e.g., a nickel titanium alloy), stainless steel, or the like. In some instances, a proximal portion and a distal portion of the elongated member 212 may be made from different materials and may be connected together. For example, the distal portion of the elongated member 212 may be made from hydrophilic material and the proximal portion of the elongated member 212 may be made from either hydrophilic or hydrophobic material. In some embodiments, the proximal and distal portions may be a unitary structure made substantially from a single material. In such instances, the elongated member 212 may be coated wholly or partially with a hydrophilic coating to reduce friction at an outer surface of the guidewire 210.

The above descriptions of the guidewire 210 are just examples. Other structures for the guidewire 210 are contemplated.

FIG. 3 is a cross-sectional view of a portion of the guidewire 210. Here, the deflectable tip 232 is shown in its undeflected position (e.g., at a position concentric about the longitudinal axis L-L). The body 234 and/or the deflectable tip 232 of the distal tip 230 may be made of one or more solid pieces of material or may be made of at least one or more partially hollow materials allowing the lumen 220 to pass therethrough.

The body 234 and the deflectable tip 232 of the distal tip 230 may be made from any biocompatible material. Illustratively, the deflectable tip 232 may be made from the same material (e.g., stainless steel, Nitinol or polymers) as the body 234. Alternatively, the deflectable tip 232 may be made of a material that is softer than a material of the body 234. In some instances the deflectable tip 232 may be made of a material that is softer than a material of the main body 224 of the guidewire 210.

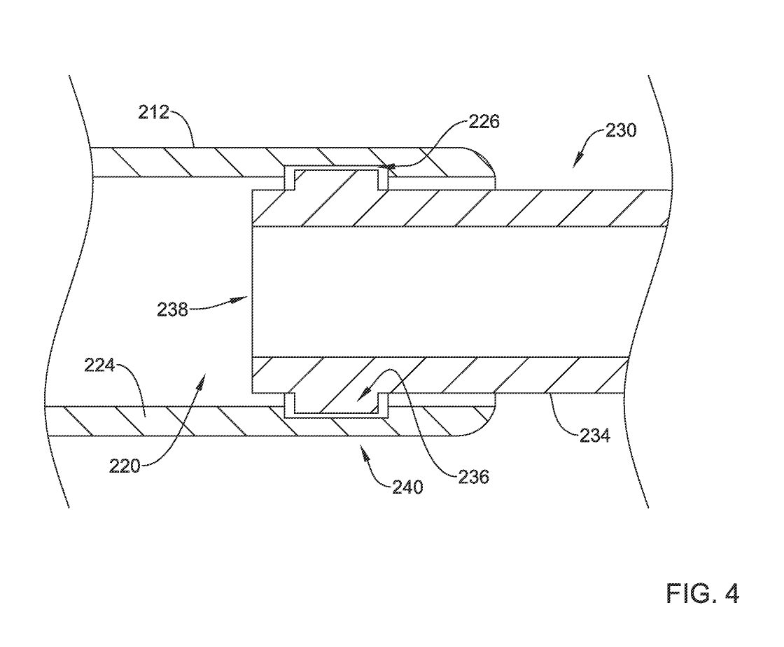

FIG. 4 illustrates a portion of an example guidewire. As shown in FIG. 4, the body 234 of the distal tip 230 may include a region 236 protruding and/or extending radially around a circumference of the body 234. The region 236 may be unitarily formed with the body 234 or connected to the body 234 with any connection technique, as desired. Illustratively, the region 236 may be located adjacent or near a proximal end 238 of the distal tip 230. A distal portion of the main body 224 (e.g., a portion of the main body adjacent to or a part of the distal end 216 of the elongated member 212) may include a recess 226 formed therein to receive the region 236 of the distal tip 230. Such a connection between the distal tip 230 and the main body 224 may form a snap-fit connection, or other connection type, between the region 236 and the recess 226 to form the connection 240. Alternatively or in addition, one or more adjustment members (e.g., a ball bearing or other member), may be utilized to facilitate rotation of the distal tip 230 with respect to the main body 224.

FIG. 5 shows side-to-side motion of the guidewire 210. The guidewire 210 may be introduced into a central lumen of a catheter, cannula, or sphincterotome 300. The guidewire 210 and the sphincterotome 300 may be inserted into a proximal portion of an endoscope shaft 302, and may be advanced through a central lumen of the endoscope shaft 302, toward the side opening 304. The sphincterotome 300 and the guidewire 210 may emerge from the opening 304, and may extend through or otherwise engage the plug/elevator 306. As the sphincterotome 300 and the distal tip 230 of the guidewire 210 extend from the opening 304, the plug 306 may be moved to facilitate positioning of the sphincterotome 300 and the guidewire 210. In one example, the plug 306 may be tilted to redirect the sphincterotome 300 and the guidewire 210 into alignment with the papilla 14. As the sphincterotome 300 and the guidewire 210 extend farther out from the opening 304, portions of the guidewire 210 may be extended from the sphincterotome 300 so that the distal tip 230 may advance toward the papilla 14.

In one instance, the distal tip 230 while traversing through the papilla 14 may move normal to or substantially normal to the longitudinal axis L-L of the distal tip 230, in a repeated side-to-side motion, as indicated by A and A′ in FIG. 5, (e.g., vibrate). Such repeated movements of the distal tip 230 may help it wiggle through the narrow passage within the papilla of Vater 14 to access the common bile duct 18 and/or the pancreatic duct 16. In some embodiments, such movements of the distal tip 230 may also be helpful in navigating past stones and lesions that may be present within the body lumen (e.g., within the papilla of Vater 14, the pancreatic duct 16, the common bile duct 18, etc.). Movement of the distal tip 230 may be designed to have an insignificant impact on a patient's body tissue to minimize damage to body tissue or other body parts that it may contact.

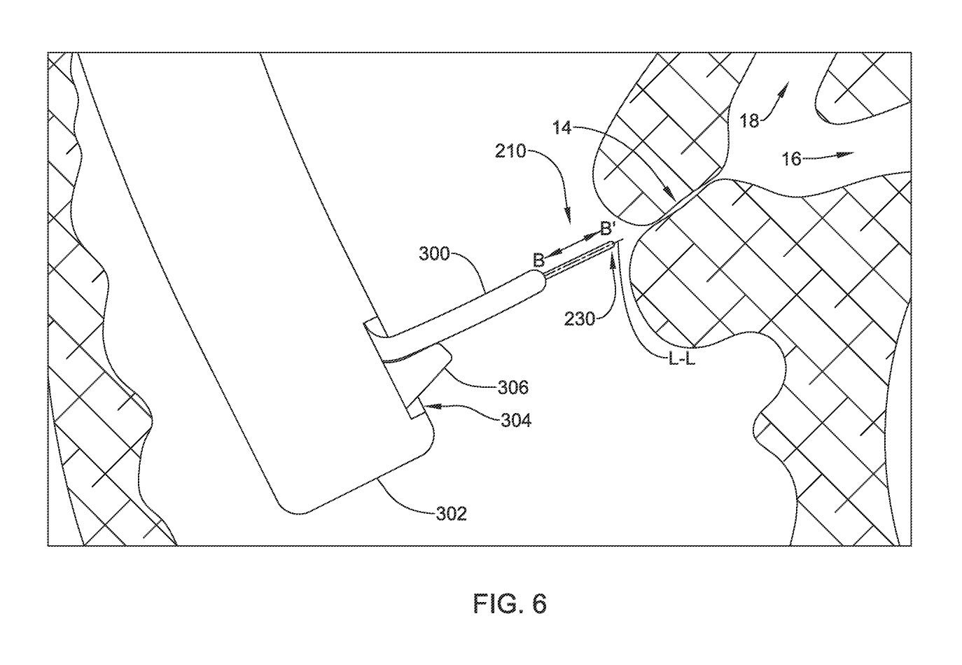

In some instances, for example as shown in FIG. 6, the distal tip 230 may undergo axial motion as indicated by a line B-B′. The distal tip 230 may move back and forth (e.g., in and out) along the longitudinal axis L-L in a direction indicated by the line B-B′. In some instances, such back and forth movement along the longitudinal axis L-L of the distal tip 230 may be longitudinal oscillation movement, which may facilitate navigation of the guidewire 210 through the papilla of Vater 14 and/or other narrow passages, while limiting the impact on a patient's body of such traversing.

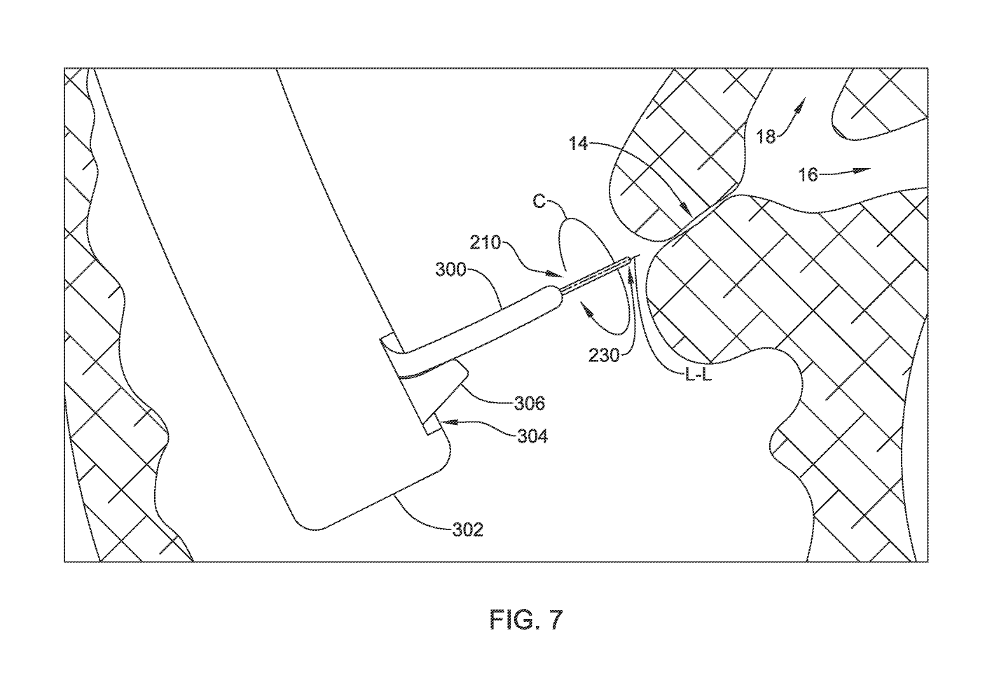

FIG. 7 shows rotational motion of the distal tip 230. The distal tip 230 may rotate around the longitudinal axis L-L in a clockwise direction C or in a counter-clockwise direction. Such rotational motion may facilitate navigating through the papilla of Vater 14 and/or other narrow passages, while limiting the impact on a patient's body of such traversing.

In some instances, the guidewire 210 may be capable of being moved in a plurality of movements simultaneously or in sequence. In one example, the guidewire 210 may be longitudinally oscillated and vibrated simultaneously or sequentially. In another example, the guidewire 210 may be longitudinally oscillated and rotated simultaneously or sequentially. In another example, the guidewire 210 may be vibrated and rotated simultaneously or sequentially. In yet another example, the guidewire 210 may be longitudinally oscillated, vibrated, and/or rotated. In some instances, the guidewire 210 may be bending while also longitudinally oscillating, vibrating, and/or rotating.

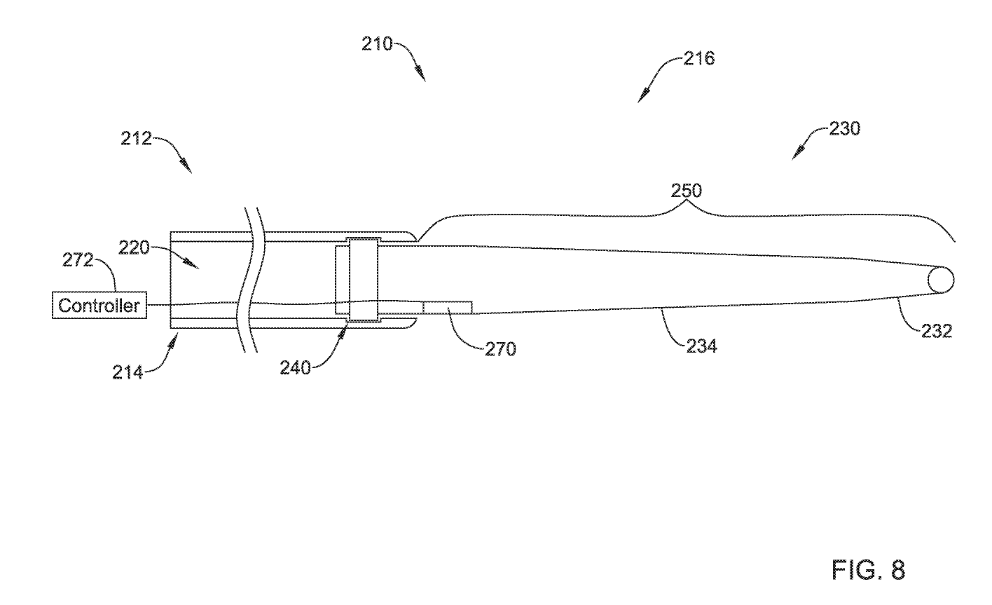

The guidewire 210 may include an electromechanical actuator 270 that may be used for actuating the movement of the distal tip 230 and/or other portions of the guidewire 210, thereby facilitating cannulation of the papilla of Vater, the common bile duct, the pancreatic duct, and/or other body lumens. As shown in FIG. 8, an electromechanical actuator 270 may be provided for actuating the movement of the distal tip 230, thereby facilitating cannulation of the common bile duct 18 or the pancreatic duct 16 (not shown in FIG. 8).

The electromechanical actuator 270 may generate mechanical movements that cause resonance within or of parts of the distal tip 230. Hence, the electromechanical actuator 270 may be employed to effect at least one of the motions such as vibration, longitudinal oscillation, and/or rotation to the distal tip 230 within or adjacent a desired duct or narrow passage. In some instances, the electromechanical actuator 270 may be a piezoelectric element, which may be attached to the elongated member 212. The piezoelectric element may be used for generation of mechanical movements that may cause motion of the distal tip 230. In some instances, the slots in the material and/or the material of the distal tip 230 may also contribute to cause resonance to its natural frequency. It is contemplated that composition and structure of the elongated member 212 may be at least partially chosen based on its resonant frequencies and the amplitude of oscillations.

In some instances, the electromechanical actuator 270 or an actuator element may be used in conjunction with a controller 272 to control the movement of the distal tip 230 of the guidewire 210. For example, the piezoelectric element may be in electrical communication with the controller 272. The controller 272 may be located at a position proximal the proximal end 214 of the guidewire 210 and the piezoelectric element may be located at one or more various locations on the guidewire 210. The controller 272 may allow for selection of one or more types of movement of the distal tip 230 such as longitudinal oscillation movement, vibration movement, rotational movement, and/or other movements. Illustratively, the controller 272 may allow for adjustment of the selected movement(s) of the distal tip 230, by controlling the frequency or amplitude of the movements.

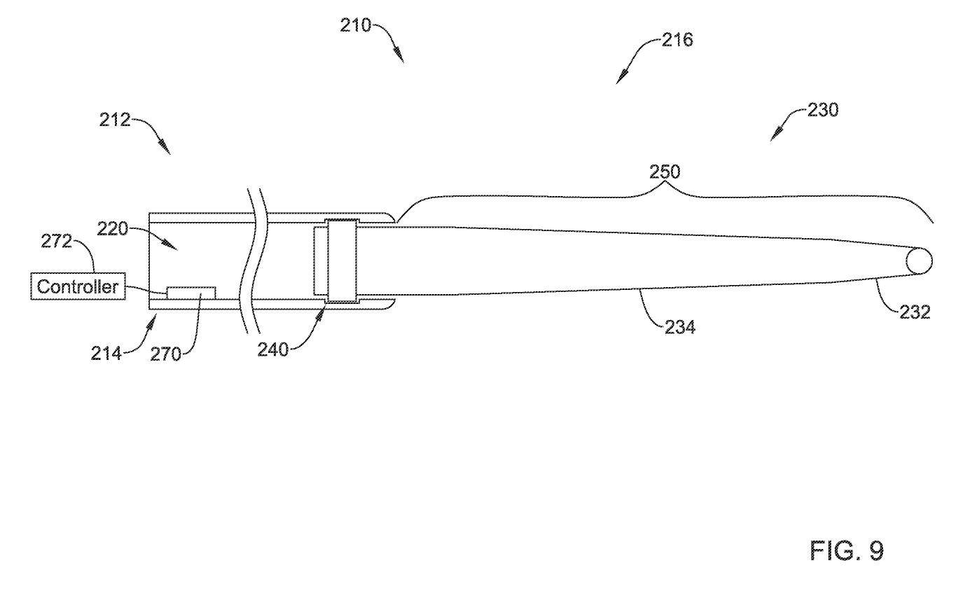

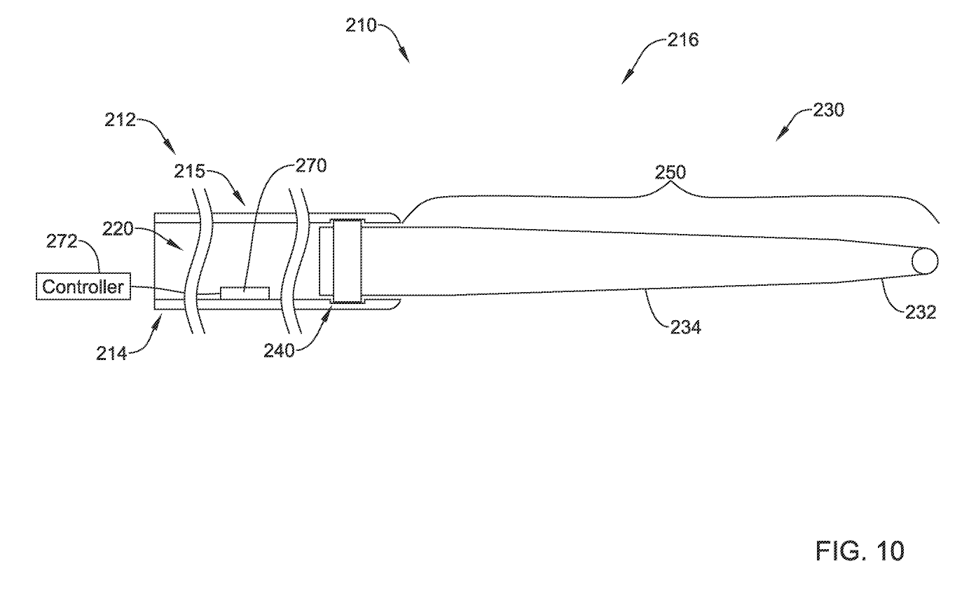

As shown in FIGS. 8-10, the electromechanical actuator 270 may be located at various locations within the guidewire 210. In some instances, the actuator element 270 may be disposed adjacent to the distal end 216 of the elongated member 212, as shown in FIG. 8. In some instances, the electromechanical actuator 270 (e.g., a piezoelectric element) may be disposed adjacent to the proximal end 214 of the elongated member 212 as shown in FIG. 9. In other instances, the electromechanical actuator 270 may be attached to a mid-portion 215 of the elongated member 212, where the mid-portion 215 is proximal to the distal end 216, as shown in FIG. 10. Such locations of the electromechanical actuator 270 may provide and/or actuate various movements of the guidewire 210 such as rotational movements, vibration movements, and/or longitudinal oscillation movements of the entire guidewire 210 or a portion thereof.

In the above embodiments of various motions of the guidewire 210, the entire guidewire 210 may undergo such motions as indicated above. Alternatively, the various motions of the guidewire 210 may be purposely substantially confined to one or more portions of the guidewire 210 (e.g., the distal tip 230, the distal end 216, the mid-portion 215, the proximal end 214, and/or other portions of the guidewire 210). In some instances, the movement of the guidewire 210 may be substantially confined to one or more portions of the guidewire 210 through selection of a position or placement of the electromechanical actuator 270 and/or through utilizing materials for the guidewire 210 with various properties to limit and/or expand the movements caused by the electromechanical actuator.

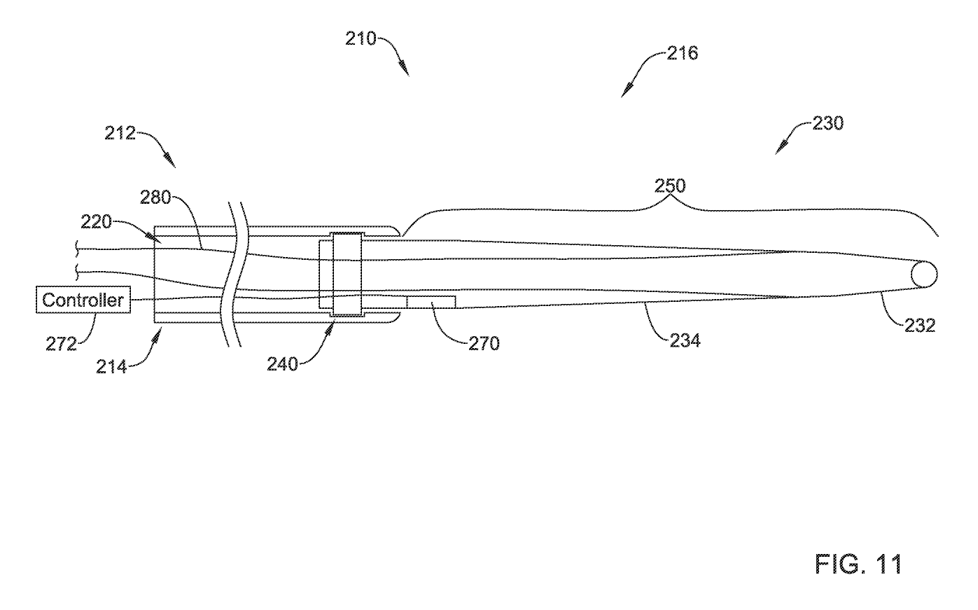

In some instances, the distal end 216 of the guidewire 210 may be steered manually or in other manners (e.g., automatically). For example, a user may be able to manually steer the distal tip 230 via pull wires 280 situated within and/or about the guidewire 210, as shown in FIG. 11. In one example, one or more pull wires 280 may be connected to the distal end 216 of the guidewire 210 and may extend through the lumen 220 to the proximal end 214 where an operator may apply force, as desired, to one or more of the pull wires 280 to steer the distal end 216 of the guidewire 210. Illustratively, the pull wires 280 may be pulled or adjusted proximally such that tension may be produced in the pull wires 280, thereby deflecting the deflectable tip 232 of the distal tip 230. In some instances, adjustment or tensioning of the pull wires 280 may steer the distal tip 230.

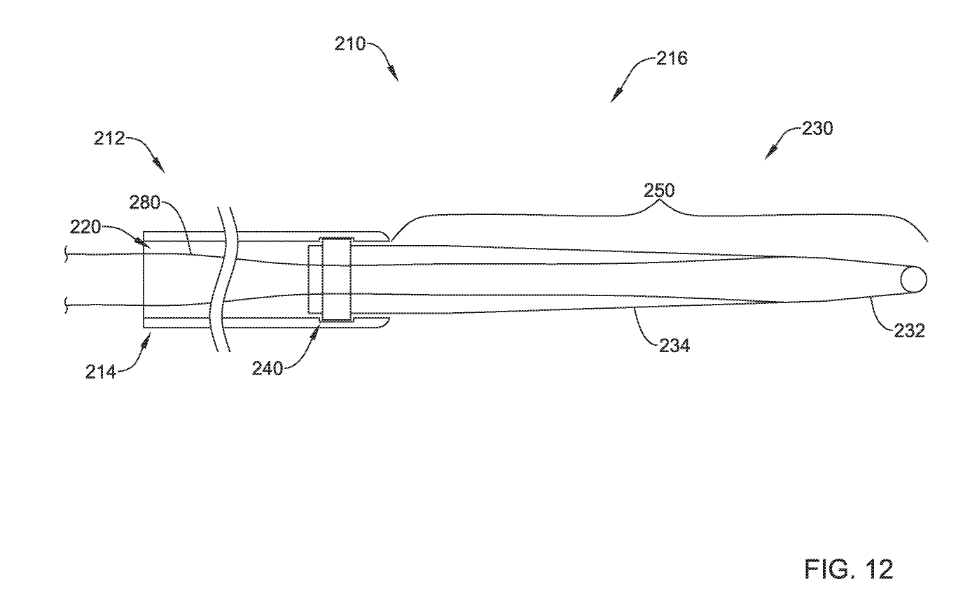

The guidewire 210 may include both the electromechanical actuator 270 for actuating movement of the distal tip 230 and a connection of the pull wires 280 for steering the deflectable tip 232 (see FIG. 11). In some instances, however, as shown in FIG. 12, the guidewire 210 may include one or two pull wires 280 for steering the distal tip 230 and may be operated/adjusted without use of the electromechanical actuator. In instances where the guidewire includes the pull wires 280, the distal tip 230 may be deflectable and may be steered toward a target duct and/or other body passage.

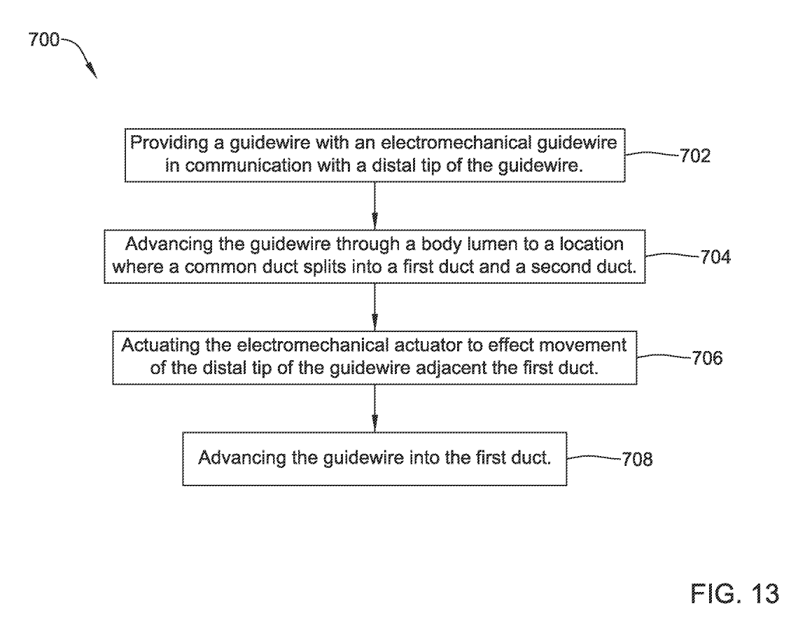

Medical devices such as the guidewires 210 described above may be used in various methods. A method 700, as shown schematically in FIG. 13, for accessing a body lumen along a biliary and/or pancreatic tract using the guidewire 210 includes a number of consecutive, non-consecutive, simultaneous, non-simultaneous, or alternative steps. In the method 700, the guidewire 210 having the electromechanical actuator 270 may be provided 702 and the electromechanical actuator 270 may be in communication with the distal tip 230 of the guidewire 210. Further, the guidewire 210 may be advanced 704 to and/or through a location where a common duct (e.g., the papilla of Vater 14) splits into a first duct (e.g., the common bile duct 18 or the pancreatic duct 16) and a second duct (e.g., the common bile duct 18 and the pancreatic duct 16). Before, during, or after advancing the guidewire 210 to a location where the common duct splits into a first duct and a second duct, the electromechanical actuator 270 may be actuated 706 to effect movement (e.g., rotation, longitudinal or axial oscillation, and/or vibration) of the distal tip 230 of the guidewire 210 adjacent to, about, and/or within the first duct. The first duct may be a desired target duct such as the common bile duct 18 or pancreatic duct 16. Then, the guidewire 210 may be advanced 708 into the first duct. In some instances, the controller 272 may be adjusted to adjust a frequency of movement or motion of the distal tip 230 adjacent, about, and/or within the first duct.

While the process steps illustrated above may provide a method for accessing a target body lumen, variations are also contemplated to these methods for achieving the same or a similar goal.

The materials that can be used for the various components of the systems presently disclosed may include those commonly associated with medical devices. For simplicity purposes, the following discussion makes reference to guidewires 210 referenced above. However, this is not intended to limit the devices and methods described herein, as the discussion may be applied to other similar devices and/or components of devices disclosed herein.

The guidewire 210 and/or components thereof may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

Some examples of suitable polymers may include, but are not limited to, polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “super elastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a super elastic alloy, for example a super elastic nitinol can be used to achieve desired properties. In at least some embodiments, portions or all of the guidewire 210 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the guidewire 210 in determining its location. Some examples of radiopaque materials can include, but are not limited to, gold, platinum, palladium, tantalum, tungsten alloy, polymer material loaded with radiopaque filler, and the like. Additionally, other radiopaque marker bands and/or coils may also be incorporated into the design of the guidewire 210 to achieve the same result.

In some embodiments, a degree of Magnetic Resonance Imaging (MRI) compatibility is imparted into the guidewire 210. For example, guidewire 210 or portions thereof may be made of a material that does not substantially distort the image and create substantial artifacts (i.e., gaps in the image). Certain ferromagnetic materials, for example, may not be suitable because they may create artifacts in an MRI image. The guidewire 210 or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

As alluded to above, the distal tip 230 and/or elongated member 212 may include one or more tubular members that may have slots formed therein. Various embodiments of arrangements and configurations of slots are contemplated. For example, in some embodiments, at least some, if not all of the slots are disposed at the same or a similar angle with respect to the longitudinal axis of the elongated member 212. The slots can be disposed at an angle that is perpendicular, or substantially perpendicular, and/or can be characterized as being disposed in a plane that is normal to the longitudinal axis of the elongated member 212. However, in other embodiments, the slots can be disposed at an angle that is not perpendicular, and/or can be characterized as being disposed in a plane that is not normal to the longitudinal axis of the elongated member 212. Additionally, a group of one or more the slots may be disposed at different angles relative to another group of one or more the slots. The distribution and/or configuration of the slots can also include, to the extent applicable, any of those disclosed in U.S. Pat. No. 7,914,467, the entire disclosure of which is herein incorporated by reference. Some example embodiments of appropriate micromachining methods and other cutting methods, and structures for tubular members including slots and medical devices including tubular members are disclosed in U.S. Pat. Publication Nos. 2003/0069522 and 2004/0181174-A2; and U.S. Pat. Nos. 6,766,720; and 6,579,246, the entire disclosures of which are herein incorporated by reference. Some example embodiments of etching processes are described in U.S. Pat. No. 5,106,455, the entire disclosure of which is herein incorporated by reference. It should be noted that the methods for manufacturing guidewire 210 may include forming the slots in the elongated member 212 using these or other manufacturing steps.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A medical guidewire for accessing a body lumen along a biliary and/or pancreatic tract, the medical guidewire comprising:
an elongated member having a distal end and a proximal end;
a movable distal tip positioned at the distal end of the elongated member;
an electromechanical actuator for causing movement of the distal tip;
wherein actuation of the electromechanical actuator actuates movement of the movable distal tip and facilitates cannulation of one or more of a common bile duct and a pancreatic duct.

2. The medical guidewire of claim 1, wherein the distal end of the elongated member is manually steerable.

3. The medical guidewire of claim 1, wherein:
the elongated member has a main body coupled to the distal tip;
the main body has a maximum first diameter and the distal tip has a maximum second diameter smaller than the maximum first diameter.

4. The medical guidewire of claim 1, wherein the electromechanical actuator comprises a piezoelectric element attached to the elongated member for effecting movement of the distal tip.

5. The medical guidewire of claim 4, wherein the electromechanical actuator comprises a controller in electrical communication with the piezoelectric element.

6. The medical guidewire of claim 5, wherein the controller allows for selection of one or more types of movement of the distal tip.

7. The medical guidewire of claim 5, wherein the controller allows adjustment of a frequency of movement of the distal tip.

8. The medical guidewire of claim 1, wherein the movement of the distal tip includes repeated movements of the distal tip.

9. The medical guidewire of claim 1, wherein the movement of the distal tip includes one or more of rotation of the distal tip, vibration of the distal tip, and longitudinal oscillation of the distal tip.

10. The medical guidewire of claim 4, wherein the piezoelectric element is disposed adjacent to the distal end of the elongated member.

11. The medical guidewire of claim 4, wherein the piezoelectric element is disposed adjacent to the proximal end of the elongated member.

12. The medical guidewire of claim 4, wherein the piezoelectric element is attached to a mid-portion of the elongated member, where the mid-portion of the elongated member is proximal the distal end of the elongated member.

13. A medical device for use with an endoscope for accessing a body lumen along a biliary and/or pancreatic tract, the medical device comprising:
an elongated member having a lumen defined therein, where the elongated member has a proximal end and a distal end;
an enabled distal tip disposed at the distal end of the elongated member;
an actuator element in mechanical communication with the distal tip to enable movement of the distal tip;
a control mechanism in electrical communication with the actuator element, where the control mechanism is capable of effecting mechanical movement of the actuator element; and
wherein adjustment of the control mechanism adjusts movement of the distal tip.

14. The medical device of claim 13, wherein the distal end of the elongated member is steerable.

15. The medical device of claim 13, wherein the movement of the distal tip is one or more of oscillation of the distal tip, rotation of the distal tip, and vibration of the distal tip.

16. A method for accessing a body lumen along a biliary and/or pancreatic tract using a guidewire having an electromechanical actuator capable of actuating movement of a distal tip of the guidewire, the method comprising:
advancing a guidewire through a body lumen to a location where a common duct splits into a first duct and a second duct, the guidewire having an electromechanical actuator in communication with a distal tip of the guidewire;
actuating the electromechanical actuator to effect movement of the distal tip of the guidewire adjacent the first duct; and
advancing the guidewire into the first duct.

17. The method of claim 16, wherein actuating the electromechanical actuator to effect movement of the distal tip of the guidewire about the first duct includes effecting rotation of the distal tip of the guidewire.

18. The method of claim 16, wherein actuating the electromechanical actuator to effect movement of the distal tip of the guidewire about the first duct includes effecting oscillation of the distal tip of the guidewire.

19. The method of claim 16, wherein actuating the electromechanical actuator to effect movement of the distal tip of the guidewire about the first duct includes effecting vibration of the distal tip of the guidewire.

20. The method of claim 16, further comprising:
adjusting a controller of electromechanical actuator to adjust a frequency of movement of the distal tip of the guidewire adjacent the first duct.
